(19) United States
(12) Patent Application Publication
(10) Pub. No.: US 2002/0142287 A1
(43) Pub. Date: Oct. 3, 2002

(54) High throughput assay to detect inhibitors of the map kinase pathway

(76) Inventors: Hirotaka Yamamoto, Glenview, IL (US); Joseph R. Moskal, Chicago, IL (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/017,178

(22) Filed: Dec. 14, 2001

Related U.S. Application Data
(60) Provisional application No. 60/255,548, filed on Dec. 14, 2000.

(51) Int. Cl. C12Q 1/00; C12Q 1/68; C12Q 1/66

(52) U.S. Cl. 435/4; 435/6; 435/8

(57) ABSTRACT

The present invention provides an assay for screening modulators of the MAPK pathway that are useful for the treatment of cancers. In addition, the present invention provides reagents and methods for detecting modulators of the MAPK pathway.

### RELATED APPLICATIONS

This application claims priority to U.S. Provisional App. No. 60/255,548 filed Dec. 14, 2000, which is incorporated by reference herein in its entirety.

### FIELD OF THE INVENTION

The present invention relates to the identification of compounds that are suitable for the treatment of cancers.

### BACKGROUND OF THE INVENTION

Many malignant tumors remain resistant to chemotherapy. Current cancer drugs are directed at a narrow range of targets, primarily inhibition of DNA synthesis or microtubule formation. The drugs currently used were developed under empirical drug discovery programs that assessed anti-proliferative activity using in vitro screening systems. These drugs do not directly target signal transduction pathways involved in cell proliferation, including the mitogen-activated protein kinase (MAPK) pathway.

Cell adhesion is involved in many different physiological mechanisms. For example, changes in cell adhesion and the remodeling of the extracellular matrix play a critical role in the invasion of malignant tumors. Extracellular-matrix degrading proteases, such as urokinase-type plasminogen activator (uPA), matrix-type metalloproteinases (MMPs), and membrane-type MMPs (MT-MMPs) have been shown to participate in the remodeling of the extracellular matrix through a proteolytic cascade. Although their biological activity is highly regulated at the post-transcriptional level, they are regulated at the transcriptional level as well. Transcription factors AP-1 and c-Ets-1 take part in the induction of uPA and several MMP genes. Mitogenic stimulation by growth factors and activation of Protein Kinase C (PKC) induces the expression of matrix degrading proteases through the induction of these transcription factors.

In particular, the role of proteolytic enzymes in glioma invasion has been studied. The expression of MMP-2, MMP-9, MT-MMP, uPA and its receptor has been found in gliomas. MMP-2 activity correlates with the invasiveness of human gliomas in vitro, and is largely dependent on post-transcriptional and post-translational modification of the enzyme. The proteolytic activation of MMP-2 by MT-MMP, and the association between MMP-2 or uPA and integrins, plays a critical role in regulating invasivity. While MMP-2 is constitutively expressed in most tumor cells, the expression of other MMPs and uPA can be transcriptionally regulated by growth factors and PKC activators through the transcription factors AP-1 and Ets-1. The MMP-1, -3, -7, -9, -10 and uPA genes contain the promoter sequence for AP-1 and PEA3.

It has been shown that the Ets family of transcription factors plays a critical role in inducing a malignant phenotype in cancer cells that have been transformed by the ras oncogene. The suppression of endogenous Ets family transcription factor(s) by the transfection of Ets transdominant mutants can reverse malignant phenotypes caused by ras-mediated transformation in NIH3T3 fibroblasts. This evidence indicates that the Ets family transcription factors are a direct downstream target affected by ras-mediated transformation. Our results indicate that further downstream target genes, which are regulated by Ets-1 transcription factor and cause malignant phenotypes, include MMP-1, MMP-3 and GnT-V in human gliomas.

### SUMMARY OF THE INVENTION

In one embodiment, the present invention provides a method for identifying a compound that affects the MAPK pathway that comprises the steps of bringing a compound into contact with a cell stably transfected with a recombinant construct comprising a polynucleotide encoding a reporter gene under the control of the c-fos promoter, followed by detecting changes in expression of the reporter gene resulting from cellular contact with the compound. In this method, a compound that affects the MAPK pathway is identified by detecting changes in expression of said reporter gene under control of the c-fos promoter. In a preferred embodiment, the reporter gene used in the method is the luciferase gene. In yet another embodiment, the method can be used to identify inhibitors of the MAPK pathway. In another embodiment, the method can be used to identify activators of the MAPK pathway. In yet another embodiment, the method utilizes a cell that constitutively expresses low levels of invasion-associated genes, whereby stimulation of said invasion-associated genes occurs via activation of the MAPK pathway. In another embodiment, the method utilizes a cell that is weakly tumorigenic, whereby c-ets-1 mRNA expression is activated exclusively via the MAPK pathway.

We have found that levels of c-Fos protein expression in human glioma SNB-19 cells correlate with MAPK pathway activity. Activation of this pathway results in induction of invasion-associated gene expression in glioma cells, while inhibition of the pathway results in suppression of both c-Fos induction and invasion-associated gene expression. Novel methods for identifying compounds that induce or inhibit MAPK pathway activity, and therefore c-Fos expression, are provided by the present invention. For example, the present invention provides for a high-throughput assay for screening modulators of the MAPK pathway. The present invention further provides reagents and methods for detecting modulators of the MAPK pathway.

In yet another embodiment, the present invention provides a cell comprising a recombinant construct responsive to modulators of the MAPK pathway, wherein said recombinant construct comprises a polynucleotide encoding a reporter gene operatively linked to the c-Fos promoter. In yet another embodiment, the reporter gene is the luciferase gene.

Other embodiments that would be understood to be within the scope of the present invention are described below.

### BRIEF DESCRIPTION OF THE DRAWINGS

FIG. 1A is a schematic illustration of the MAPK pathway and its role in the induction of the invasion-associated genes. FIG. 1B illustrates the c-fos promoter/luciferase gene reporter system of the present invention.

FIG. 2 shows the expression of N-acetylglucosaminyltransferase V (GnT-V), c-ets-1 and uPA mRNA in human tumor cell lines. 20 μg of total RNA per lane were used for Northern analysis. Lanes 1-6: human glioma cell lines, SW1088, D-54MG, U-373MG, U-87MG, U-118MG, and SNB-19, respectively. Lanes 7-10: human neuroblastoma cell lines, LAN-5, IMR-32, SKN-SH, and SKN-MC, respectively. Levels of GnT-V mRNA and c-ets-1 mRNA expression are correlated (panels A & B). Levels of uPA mRNA and c-ets-1 mRNA expression are correlated in glioma cell lines, but not in neuroblastoma cell lines (panels B & C). Ethidium bromide staining of total RNA (panel D) provides an indication of the relative amount of RNA loaded in each lane.

FIG. 3 shows the expression of MMPs in human brain tumor lines. 20 μg of total RNA per lane were use for Northern analysis. Lanes 1-6: human glioma cell lines, SW1088, D-54MG, U-373MG, U-87MG, U-118MG, and SNB-19, respectively. Lanes 7-10: human neuroblastoma cell lines, LAN-5, IMR-32, SKN-SH, and SKN-MC, respectively. MMP-1 mRNA is expressed in SW1088, U-87MG, and U-118MG glioma cell lines and SKN-SH neuroblastoma cell lines (FIG. 3A), while MMP-3 MRNA is expressed in SW1088, D-54MG, U-87MG, U-118MG glioma cell lines and SKN-SH neuroblastoma cell lines (FIG. 3B). MMP-10 MRNA expression was found only in SW1088, U-87MG, and U-118MG glioma cells (FIG. 3C). Ethidium bromide staining of total RNA is shown (FIG. 3D). Expression of MMP RNA is found in cell lines with high c-ets-1 mRNA expression, but levels of MMP expression are not correlated with the levels of c-ets-1 expression.

FIG. 4 shows the concomitant induction of GnT-V, c-ets-1, uPA, MMP-1 and MMP-3 mRNA by Phorbol 12, 13-dibutyrate (PDBu), and suppression of PDBu-mediated induction by a MAPKK inhibitor. SNB-19 cells were incubated with 150 nM PDBu for 0, 6, 12, 18, and 24 hr, and the cells were harvested for Northern analysis; 20 μg of total RNA per lane were used. Lanes 1-5: SNB-19 cells incubated with 150 nM PDBu for 0, 6, 12, 18, and 24 hr, respectively. Lane 6-7: SNB-19 cells incubated with 150 nM PDBu for 24 hr in the presence of 15 and 80 μM MAPKK inhibitor, 2′-amino-3′methoxyflavone (PD98059), respectively. FIG. 4A-4E shows mRNA expression of GnT-V, c-ets-1, uPA, MMP-1, and MMP-3, respectively. FIG. 4F shows total RNA staining by ethidium bromide. The marked induction of GnT-V, c-ets-1, uPA, MMP-1, and MMP-3 mRNA expression was detected after 24 hr incubation in the presence of 150 nM PDBu. This induction was completely abolished by the addition of the MAPKK inhibitor.

FIG. 5 shows the coordinated induction of c-Fos, c-ets-1, GnT-V, uPA, and MMP-3 mRNA by growth stimulation. Quiescent SNB-19 cells were plated into fresh growth media and incubated for 0.5, 1, 2, 3, 4, 6, 8, and 24 hr, and the cells were harvested for Northern analysis. 10 μg of total RNA per lane were used. Maximal induction of c-fos mRNA was achieved after 1 hr incubation, followed by c-ets-1 mRNA (maximal induction at 3-4 hr), then GnT-V and uPA (around 6 hr) followed by MMP-3 (8 hr). Unlike PDBu-mediated induction, MMP-1 mRNA was only weakly induced.

FIG. 6 shows the suppression by PD98059, a MEK/MAPKK inhibitor, of c-fos, c-ets-1, GnT-V, and uPA mRNA expression. Quiescent SNB-19 cells were plated into fresh growth media and incubated for 0.5, 1, 2, 3, 4, 6, and 8 hr without an inhibitor (control), or in the presence of 80 μM PD98059. 10 μg of total RNA per lane were used for Northern analysis.

FIG. 7 shows that growth stimulation induces Fos, but not Ets-1 protein expression in SNB-19 glioma cells. Quiescent SNB-19 cells were plated into fresh growth media and incubated for 0.5, 1, 2, 3, 4, 6, 8, and 24 hr, and the cells were harvested for c-Fos protein analysis by Western blot. 20 μg of total protein per lane was used. Maximal induction of c-Fos protein expression was observed at 2 hr after plating.

FIG. 8 is a schematic presentation of inhibitors of the MAPK pathway. Both PD98059 and U0126 inhibit activation (phosphorylation) of MEK/MAPK Kinase (MAPKK). U0126 is a 30 times more potent inhibitor than PD98059. Farnesyltransferase inhibitor I (FTI) inhibits farnesylation of Ras and prevents Ras from maturing into a biologically active membrane-associated form.

FIG. 9 shows inhibition of c-Fos protein induction by inhibitors of the MAPK pathway. Quiescent SNB-19 cells were plated into fresh growth media and incubated for 0.5, 1, 2, 3, 4, 6, and 8 hr in the presence of inhibitors of the MAPK pathway, and the cells were harvested for c-Fos protein analysis by Western blot. 20 μg of total protein per lane were used. c-Fos protein induction without inhibitors was shown as a control. Inhibitors of MAPK pathway suppress Fos protein induction. Potency of inhibition is 50 μM U0126>1 μM FTI>50 μM PD98059.

FIG. 10 shows that the MEK/MEPKK inhibitor U0126 suppressed the induction of GnT-V, uPA and c-ets-1 mRNA expression and inhibited phosphorylation of ERK/MAPK. Quiescent SNB-19 cells were plated into fresh growth media and incubated for 0.5, 1, 2, 3, 4, 6, and 8 hr, and the cells were harvested for Northern analysis (FIG. 10A-10C) and Western blot analysis (FIG. 10D). 50 μM U0126 strongly suppressed induction of GnT-V (FIG. 10A), uPA (FIG. 10B), c-ets-1 (FIG. 10C), and completely inhibited phosphorylation of p42/p44 ERK/MAPK proteins, downstream substrates of MEK/MAPKK (FIG. 10D).

FIG. 11 shows that phosphatidyl inositol 3-kinase (PI 3-Kinase) and epidermal growth factor-receptor (EGF-R) kinase have little effect on the expression of GnT-V, uPA and c-ets-1 mRNA induced by growth stimulation. Quiescent SNB-19 cells were plated into fresh growth media and incubated for 2 and 6 hr, and the cells were harvested for Northern analysis. 20 μg of total RNA per lane were used. PI 3-kinase inhibitor (Wortmannin) and EGF-R kinase inhibitor (PD153035) showed no effect on invasion-associated gene expression.

FIG. 12 shows the stable transfection of the luciferase gene under control of the fos promoter in SNB-19 glioma cells. Three quiescent transfectants were plated into fresh growth media and incubated for 0.5, 1, 2, and 7 hr, and the cells were harvested for detection of c-Fos and luciferase mRNA expression by Northern analysis. 10 μg of total RNA per lane were used. Growth-stimulated induction of luciferase mRNA expression was observed at 0.5 hr, similar to c-Fos mRNA, and lasted for a longer period than c-Fos MRNA expression.

FIG. 13 shows the inhibition of luciferase gene expression by MEK/MAPK inhibitors, PD98059 and U0126, in a stable transfectant. A quiescent c-fos promoter/luciferase gene transfectant (clone 60) was plated into fresh growth media and incubated for 1, 2, 3, 6, and 24 hr in the presence of MEK/MAPKK inhibitors, and the cells were harvested for detection of luciferase mRNA expression by Northern analysis. 10 μg of total RNA per lane were used. Luciferase mRNA expression was suppressed by the inhibitors. Strong MEK/MAPPK inhibitor U0126 greatly suppressed luciferase gene expression, while weak inhibitor PD98059 slightly suppressed the gene expression compared to the control.

### DETAILED DESCRIPTION OF THE INVENTION

Within this application, unless otherwise stated, definitions of the terms and illustration of the techniques of this application may be found in any of several well-known references, such as: Sambrook, J., et al., Molecular Cloning: A Laboratory Manual, Cold Spring Harbor Laboratory Press (1989); Goeddel, D., ed., Gene Expression Technology, Methods in Enzymology, Vol. 185, Academic Press, San Diego, Calif. (1991); “Guide to Protein Purification”in Deutshcer, M.P., ed., Methods in Enzymology, Academic Press, San Diego, Calif. (1989); Innis, et al., PCR Protocols: A Guide to Methods and Applications, Academic Press, San Diego, Calif. (1990); Freshney, R. I., Culture of Animal Cells: A Manual of Basic Techniques, 2^{nd} ed., Alan Liss, Inc. New York, N.Y (1987); Murray, E.J., ed., Gene Transfer and Expression Protocols, pp 109-28, The Humana Press Inc., Clifton, N.J. and Lewin, B. Genes VII, Oxford University Press, New York (2000). All references cited within this application are hereby incorporated by reference.

As used herein, a “transcriptional regulatory region” is defined as any region of a gene involved in regulating transcription of a gene, including, but not limited to, promoters, enhancers, and repressors. A “transcriptional regulatory element” is defined as any element involved in regulating transcription of a gene, including, but not limited to, promoters, enhancers, and repressors. A “promotor” is a regulatory sequence of DNA that is involved in the binding of RNA polymerase to initiate transcription of a gene. A “gene” is a segment of DNA involved in producing a peptide, polypeptide, or protein, including the coding region, non-coding regions preceding (“leader”) and following (“trailer”) the coding region, as well as intervening non-coding sequences (“introns”) between individual coding segments (“exons”). “Coding” refers to the representation of amino acids, start and stop signals in a three base “triplet” code. Promoters are often upstream (“5′ to ”) the transcription initiation site of the corresponding gene. Other regulatory sequences of DNA in addition to promoters are known, including sequences involved with the binding of transcription factors, including response elements that are the DNA sequences bound by inducible factors. “Enhancers” comprise yet another group of regulatory sequences of DNA that can increase the utilization of promoters, and can function in either orientation (5′-3′ or 3′-5′) and in any location (upstream or downstream) relative to the promoter. Preferably, the regulatory sequence has a positive activity, i.e., binding of an endogenous ligand (e.g., a transcription factor) to the regulatory sequence increases transcription, thereby resulting in increased expression of the corresponding target gene. The term “operably linked” or “functionally linked” refers to the combination of a first nucleic acid fragment representing a transcriptional control region having activity in a cell joined to a second nucleic acid fragment encoding a reporter or effector gene such that expression of said reporter or effector gene is influenced by the presence of said transcriptional control region.

A “responsive element” is a portion of a transcriptional control region that induces expression of a nucleotide sequence. There may be multiple responsive elements within a single transcriptional control region and each of these elements may function independently of any other elements of the transcriptional control region. Thus, a responsive element may be incorporated into a reporter gene vector independent from the remainder of the transcriptional control region from which it is derived and function to drive expression of the reporter gene under the proper conditions.

“Cancer” is defined herein as any cellular malignancy for which a loss of normal cellular controls results in unregulated growth, lack of differentiation, and increased ability to invade local tissues and/or metastasize. Cancer may develop in any tissue of any organ at any age. Cancer may be an inherited disorder or caused by environmental factors or infectious agents; it may also result from a combination of these. For the purposes of utilizing the present invention, the term cancer includes both neoplasms and premalignant cells.

“Brain cancer” is defined herein as any cancer involving a cell present in the nervous system. Examples of brain cancers include, but are not limited to, intracranial neoplasm such as those of the skull (e.g., osteoma, hemangioma, granuloma, xanthoma, osteitis deformans), the meniges (e.g., meningioma, sarcoma, gliomatosis), the cranial nerves (e.g., glioma of the optic nerve, schwannoma), the neuroglia (e.g., gliomas, including oligodendroglioma and glioblastoma multiforme (GBM)) and ependyrna (e.g., ependyrnomas), the pituitary or pineal body (e.g., pituitary adenoma, pinealoma), and those of congenital origin (e.g., neuroblastoma, medulloblastoma, craniopharyngioma, chordoma, germinoma, teratoma, dernoid cyst, angioma, hemangioblastoma) as well as those of metastatic origin.

“Invasion-associated gene” is defined as a gene that is either over-expressed or under-expressed in tumor cells that are invasive. Gene products of invasion-associated genes can play a role in the invasion of malignant tumors. Examples of invasion-associated genes include, but are not limited to, the matrix-metalloproteinases (MMPs), including MMP-1, MMP-3, and MMP-10, urokinase-type plasminogen activator (uPA), N-acetylglucosaminyltransferase V (GnT-V), and the transcription factor c-Ets-1.

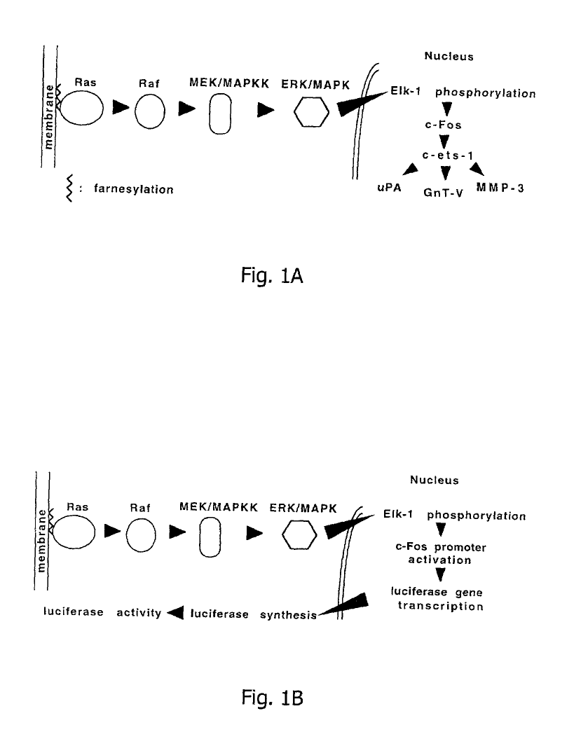

The “MAPK pathway,” shown schematically in FIG. 1A, is an intracellular signal-transduction pathway comprising a kinase cascade that produces the activation of mitogen-activated protein kinase (MAPK).

In one embodiment, the present invention provides a recombinant DNA construct comprising a transcriptional regulatory region, promoter, or other regulatory element (e.g., enhancer) of the c-Fos gene. For example, the present invention provides a recombinant DNA molecule comprising a transcriptional control region of c-Fos operably linked to a reporter gene such as luciferase (LUC), β-galactosidase (β-gal), green fluorescent protein (GFP), red fluorescent protein (RFP), yellow fluorescent protein (YFP), chloramphenical acetyl transferase (CAT), or other reporter gene. Many other reporter genes are also available to the skilled artisan that would be suitable for the purposes of this invention.

Preferably, the transcriptional regulatory region, promoter, or other regulatory element from c-Fos is operably linked to a nucleotide sequence encoding the reporter protein in an expression vector construct. In certain embodiments, at least a portion of the 3′-untranslated region of c-fos is included that is sufficient to cause the time course of the loss of the reporter MRNA to correspond to the time course of c-fos mRNA expression.

The construct is transiently or stably transfected into a cell. The cell can then be exposed to a treatment protocol (e.g., a potentially chemotherapeutic compound) that affects, by activating or inhibiting, the activity of the transcriptional regulatory region, promoter or regulatory element, resulting in increased or decreased expression of the reporter gene within the cell.

Expression of the reporter gene is determined by detection of the reporter protein by, for instance, a luciferase assay where the reporter sequence encodes luciferase. Many types of reporter gene assays are available to the skilled artisan. In this manner, a system is provided whereby the potential influence of a particular condition or compound on gene expression in a cell is determined. In addition, the system can be configured to provide a high-throughput assay for identifying compounds that increase or decrease expression of genes involved in cancer. As such, the method is useful for drug discovery and drug safety evaluations.

In another embodiment, the present invention provides a cell for use in identifying compounds that affect the MAPK pathway. In a preferred embodiment, the cell has the characteristic that induction of c-Fos protein and c-fos mRNA is specific to the activation of the MAPK pathway. In another preferred embodiment, the cell has the characteristic that growth-stimulated induction of the invasion-associated genes in the cell occurs via activation of the MAPK pathway. In yet another preferred embodiment, the cell is weakly tumorigenic, whereby activation of c-ets-1 mRNA expression occurs exclusively via the MAPK pathway. In another preferred embodiment, the cell is a SNB-19 glioma cell. For example, weakly tumorigenic SNB-19 glioma cells constitutively express low levels of the invasion-associated genes, and growth stimulation in these cells induces invasion-associated genes via the activation of the MAPK pathway. On the other hand, highly tumorigenic U-87MG constitutively express high levels of invasion-associated genes, and can utilize alternative signaling mechanisms to activate c-ets-1 mRNA expression.

Compounds identified with the current invention will be useful for treatment of cancerous cells other than the cells of the invention. For example, highly tumorigenic U-87MG glima cells can utilize signaling mechanisms other than the MAPK pathway to activate c-ets-1 mRNA expression. In U-87MG glioma cells, however, inhibitors of the MAPK pathway can suppress expression of uPA, MMP-1, GnT-V, and α3 integrin mRNA. α3 integrin is the predominant integrin expressed in malignant gliomas and α3 integrin mRNA expression is quantitatively related to the grade of malignancy in both gliomas and medulloblastomas in clinical specimens. Transcriptional control of these invasion-associated genes is highly complex and mediated by multiple mechanisms, yet activation of the MAPK pathway plays a major role in the expression of uPA, MMP-1, GnT-V, and α3 integrin mRNA in glioma cell lines. Thus, activation of the MAPK pathway induces proliferative signaling as well as cellular invasion signaling via increased expression in both the expression of α3β integrin and aberrant N-glycosylation, and in the matrix-degrading proteinases. For example, it has been shown that alteration of α3β integrin glycosylation by α2,6-sialyltransferase gene transfection led to changes in cell-extracellular interactions and inhibited glioma formation in vivo (Yamamoto, H., et al., α2,6-Sialylation of cell-surface N-glycans inhibits glioma formation in vivo. Cancer Res., 61: 6822-29, 2001). Therefore, compounds that modulate the MAPK pathway, as identified by the present invention, are useful for treatment of, among others, highly tumorigenic cells.

The following Examples are for illustrative purposes only and are not intended, nor should they be construed as limiting the invention in any manner. Those skilled in the art will appreciate that variations and modifications can be made without violating the spirit or scope of the invention.

### EXAMPLES

### Example 1

### Expression of GnT-V, c-ets-1, uPA, and MMPs in Human Brain Tumor Cell Lines

A. Materials and Methods

Cell Culture and Brain Tumor Specimens. All established human brain tumor cell lines were maintained using Dulbecco's modified Eagle's medium (DMEM, containing 4.5 g/L glucose) supplemented with 10% heat-inactivated fetal bovine serum (FBS) (Whittaker BioProducts, Walkersville, Md.). The following cell lines were used for analysis: Human glioblastoma, SNB-19 and D-54MG (generously provided by Dr. Paul Komblith, Univ. of Pittsburgh and Dr. Darrell Bigner, Duke University, respectively); Human glioblastomas, U-87MG, U-373MG, U-118MG, and SW1088 (American Type Culture Collection (ATCC), Rockville, Md.); Human neuroblastoma cell lines, SKN-SH, SKN-MC, and IMR (ATCC), and LAN-5 (generously provided by Dr. Stephan Ladish, Children's Research Institute, Washington, D.C.); Human hepatocarcinoma, Hep G2 (ATCC) as a positive control for GnT-V.

c-ets-1 cDNA and Probes. Human c-ets-1 cDNA was cloned using RT-PCR and poly A+ RNA from U-87MG cells based on the sequence reported previously (Wasylyk, B., et al., The c-ets proto-oncogenes encode transcription factors that cooperate with c-Fos and c-Jun for transcriptional activation. Nature (London), 346: 191-93, 1990). A sense primer 5′-TTGGGAAGAAAGTCGGATT-3′(SEQ ID No. 1) (bp -119 to -101) and an antisense primer 3′-CAGGCTGAATTCATTCACAGC-5′ (SEQ ID No. 2) (bp 270 to 250) were used for RT-PCR under standard conditions (35 cycles of 96° C. denaturation, (30 seconds)/55° C. annealing (60 seconds)/72° C. polymerization (60 seconds), and a final extension at 72° C. for seven minutes, hold at 435 cycles 4° C.). A 398 bp PCR product was cloned into pT7 Blue T vector (Novagen, Madison, Wis.) and the sequence of the insert was confirmed by the dideoxy termination method (Sequenase, United State Biochemical, Cleveland, Ohio). The c-ets-1 cDNA insert was isolated from the gel after Nde I and Bam HI digestion and was used as a cDNA probe.

GnT-VcDNA and Probes. A 1.24 kb human GnT-V cDNA (GenBank Accession No. D17716) was isolated after EcoR I restriction digestion and was also used as a cDNA probe for Northern analysis.

uPA cDNA and Probes. Human uPA cDNA was cloned using RT-PCR and polyA+ RNA isolated from U-87MG glioma cells based on the sequence reported previously (GenBank Accession No. A18397). Primer A, TTGTTGTGTGGGCTGTGAGT (SEQ ID No. 3) and Primer B, ACTGGCCAAGAAAGGGACAT (SEQ ID No. 4) were used for RT-PCR under standard conditions (35 cycles of 96° C. denaturation, (30 seconds)/55° C. annealing (60 seconds)/72° C. polymerization (60 seconds), and a final extension at 72° C. for seven minutes, hold at 435 cycles 4° C.). A 408 bp PCR product was cloned into pCR2.1 vector (Invitrogen, Carlsbad Calif.) and the sequence of the insert was confirmed. The cDNA insert was isolated from the agarose gel after EcoR I restriction digestion and was used as a cDNA probe for Northern analysis.

MMP cDNA and Probes. Human MMP cDNAs expressed in human glioma cell lines were cloned by using the reverse-transcriptase-polymerase chain reaction (RT-PCR). Poly A⁺ RNA was isolated from D-54MG and U-373MG human glioma cell lines and used for RT-PCR. Based on the consensus sequences among MT-MMPs and MMPs, degenerate MMP primers, a sense degenerate primer 5′-GTG/TGCT/AGT/CC/ITCATTGGCCAC-3′ (SEQ ID No. 5) and an antisense degenerate primer 5′-GGC/AAGDG/CC/AYYGCCA-3′ (SEQ ID No. 6), were used for the PCR under standard conditions (35 cycles of 96° C. denaturation, (30 seconds)/55° C. annealing (60 seconds)/72° C. polymerization (60 seconds), and a final extension at 72° C. for seven minutes, hold at 435 cycles 4° C.). Within the degenerate oligonucleotides, a “/” between two bases indicates the site is degenerate for the bases on either side of the “/” (i.e., G/T indicates G or T occupies that position), “Y” represents a C or T and “D” represents A, G or T. The PCR-amplified product (about 400 base pairs) was subcloned into pCR2.1 vector (Invitrogen, Carlsbad, Calif.), and the cDNA insert of the individual clones were sequenced. Clones were identified that contained sequence corresponding to MMP-1, MMP-3, and MMP-10, and these were used for Northern analysis.

Procedure for Northern Blot. Surgical specimens were immediately frozen in liquid nitrogen upon resection. Total RNA was isolated from clinical glioma specimens and cultured brain tumor cells using guanidium isothiocyanate followed by CsCGl₂ centrifugation. 10-20 μg of total RNA per tumor cell line per lane were electrophoresed in an agarose-formaldehyde gel and transferred to Duralon nylon membranes (Stratagene, La Jolla, Calif.). After UV cross-linking, the blots were hybridized with a ³²P-radiolabeled cDNA probe synthesized by using a random priming kit (Stratagene, La Jolla, Calif.) and ExpressHyb solution (Clontech, Palo Alto, Calif.). The blots were then exposed to X-OMAT film (Kodak, Rochester, N.Y.) and the films were developed appropriately.

Procedure for Western Blot. To detect c-Ets-1 and c-Fos protein expression in brain tumor cell lines, 20 μg of protein cell lysates were loaded on a 8% SDS-polyacrylamide gel immediately after boiling each sample in the presence of 2% mercaptoethanol. After electrophoresis, proteins were transferred to a PVDF membrane and the membrane was blocked with 5% BSA in PBS. The membrane was then incubated with either a 1:10,000 dilution of monoclonal anti-human c-Ets-1 antibody (Clone 47, Transduction Laboratory, Ky.) or anti-human c-Fos antibody (Santa Cruz Biotechnology, Inc.) in Tris-buffered saline pH 7.4 (TBS) containing 2% BSA and 0.1% Tween 20 for 1 h at room temperature. The membrane was then washed with TBS containing 2% BSA and 0.1% Tween 20 for 10 min, followed by washing twice with 0.1% Tween 20 in TBS. Next, the membrane was incubated with a 1:10000 dilution of horseradish peroxidase-conjugated anti-mouse IgG (Amersham, UK) for 1 h at room temperature in 2% BSA in TBS containing 0.1% Tween 20. The membrane was then washed as described above, and developed with the ECL Chemiluminescence detection system (Amersham, UK) according to manufacturer's instructions.

This skilled artisan would understand this technique to be applicable using another antibody, such as an antibody reacting with GnTV, by interchanging the ets-1 antibody with the GnTV antibody. Antibodies to other proteins of interest may also be utilized. For example, an antibody to uPA is available from American Diagnostica (Greenwich, Conn.; product number 3689/398), MMP-1 antibodies are available from Lab Vision Corporation (Freemont, Calif.; see, for example, clone X2A, clone VI3, clone COMY 4A2, clone III7, and/or clone III12B), and MMP-3 antibodies are available from Lab Vision Corporation (Freemont, Calif.; see, for example, clone SL-1 ID3, clone SL-1 ID4, clone SL-1 IIIC4, and/or clone SL-1 IVB1).

B. Expression of GNT-V, ets-1, and uPA

It is important to be able to identify compounds that have an effect on glioma invasion, both to identify compounds that can be used to treat this form of cancer and to identify compounds that will increase the incidence of glioma invasion, and therefore will not be suitable for treatment of a patient. It is therefore advantageous to identify genes whose expression is correlated to glioma invasion.

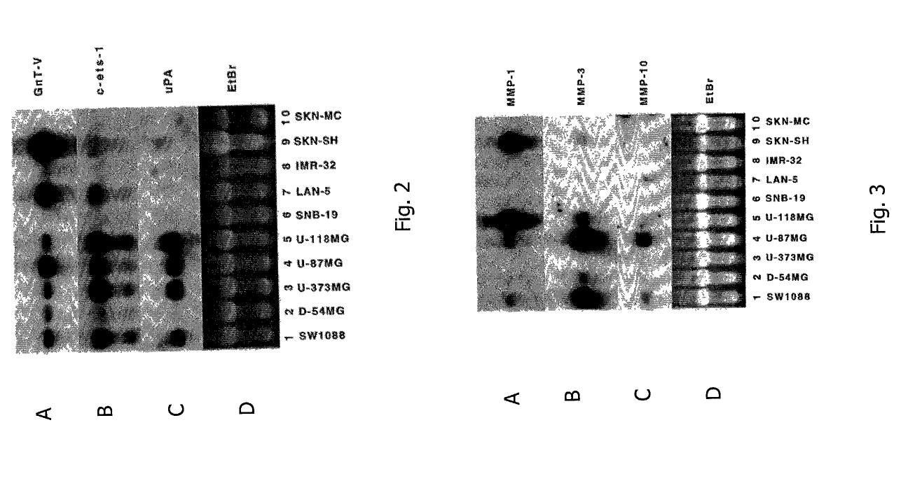

GnT-V, c-ets-1, and uPA MRNA expression was studied in a panel of six glioma and four neuroblastoma human brain tumor cell lines. Eight of ten cell lines expressed both GnT-V and c-ets-1 mRNA, and the levels of gene expression were well correlated. Those cell lines with high levels of GnT-V mRNA expression showed strong c-ets-1 mRNA expression, while the SNB-19 glioma and SKN-MC neuroblastoma cell lines showed very low expression of both GnT-V and c-ets-1 mRNA (FIG. 2 and Table 1). In addition, the levels of uPA MRNA expression is correlated with the level of c-ets-1 mRNA expression in glioma cell lines, although they were not correlated in neuroblastoma cell lines (FIG. 2 and Table 1). A previous western blot had shown that the 51 kDa Ets-1 protein was expressed uniformly in the entire panel of brain tumor cell lines examined.

B. Expression of MMPs.

Matrix-metalloproteinases (MMPs) are involved in the remodeling of the extracellular matrix, which plays a critical role in the invasion of malignant tumors. mRNA expression MMPs was examined in a panel of human brain tumor cell lines. Both MMP-1 and MMP-3 were expressed in SW-1088, U-87MG and U-118 glioma cell lines and in SKN-SH neuroblastoma cells, while D54-MG glioma cells expressed low levels of MMP-3 with no MMP-1 expression (FIGS. 3A and 3B). Neither MMP-1 nor MMP-3 was expressed in U-373 MG or SNB-19, and three other neuroblastoma cell lines showed no MMP expression. MMP-10 expression was found in SW1088 and U-87MG glioma cell lines (FIG. 3C), which express high GnT-V MRNA. A low level of MMP-10 expression was also found in U-118MG, which expresses a lower level of GnT-V mRNA than either SW1088 or U-87MG. No MMP-10 mRNA expression was found in neuroblastoma cell lines. MMP-1 and MMP-3 MRNA expression was primarily associated with cell lines which showed high c-ets-1 expression, except in U-373MG glioma and LAN-5 neuroblastoma cell lines. No MMP mRNA expression was found in SNB-19 glioma or IMR32 and SKN-MC neuroblastoma cell lines, all of which expressed little c-ets-1 mRNA. Expression of MMP MRNA is found in cell lines with high c-ets-1 MRNA expression, but unlike GnT-V mRNA expression, the levels of MMP expression were not well correlated with the levels of c-ets-1 MRNA expression in those cell lines.

TABLE 1
Expression of Ets-1, GnT-V, MMP-1,
MMP-3 and MMP-10 mRNA in Cell Lines¹

| Cell Line | Cell-type | MMP-1 | MMP-3 | MMP-10 | GnT-V | Ets-1 | uPA |
|---|---|---|---|---|---|---|---|
| SW1088 | Glioma | + | + | + | + | + | + |
| U87MG | Glioma | + | + | + | + | + | + |
| U118 | Glioma | + | + | ˜+ | + | + | + |
| D54MG | Glioma | − | + | − | + | + | ˜+ |
| U373MG | Glioma | − | − | − | + | + | + |
| SNB-19 | Glioma | − | − | − | − | ˜+ | + |
| LAN-5 | Neuro- | − | − | − | + | + | − |
| | blastoma | | | | | | |
| IMR32 | Neuro- | − | − | − | + | ˜+ | − |
| | blastoma | | | | | | |
| SKNMC | Neuro- | | | − | − | − | − |
| | blastoma | | | | | | |
| SKNSH | Neuro- | + | + | − | + | + | ˜+ |
| | blastoma | | | | | | |

¹“+” indicates high level expression. “˜+” indicates low level expression. “−” indicates very low level to absence of expression.

### Example 2

### An Assay System to Identify Compounds that Modulate Expression of c-ets-1, GnT-V, MMP-1 and MMP-3 in Cancer Cells

A. Assay for Identification of Compounds that Induce Gene Expression

An assay system has been developed that can screen for compounds that may modulate tumor cell activity, including invasion, in a patient. This assay utilizes the fact that c-ets-1, GnT-V, MMP-1, and MMP-3 gene expression is correlated to glioma invasion. Previously, c-Ets-1 has been shown to control GnT-V transcription in human bile duct carcinoma HuCC-T1 cells (Alessi, D.R., et al., PD 098059 is a specific inhibitor of the activation of mitogen-activated protein kinase kinase in vitro and in vivo. J Biol. Chem., 270: 27489-94, 1995) and c-ets-1, MMP-1 and MMP-3 mRNA expression has been shown to be modulated by growth factors and PKC activators, such as phorbol ester, in human fibroblast cells. Therefore, a PKC activator, such as a phorbol ester, was a good candidate to determine the efficacy of the assay.

The screening assay was developed to identify compounds that modulate expression of a panel of nucleic acids in a cancer cell. It is not necessary that each sequence in the panel be assayed. It is possible, for example, that analysis of c-ets-1 expression alone would be sufficient. In this example, the panel of sequences comprises ets-1, GnT-V, uPA, MMP-1, and MMP-3. Resting SNB-19 glioma cells were utilized for the model system because expression of MMPs, GnT-V or ets-1 mRNA is virtually absent (FIGS. 2 and 3). Low levels of uPA expression are observed in SNB-19 cells.

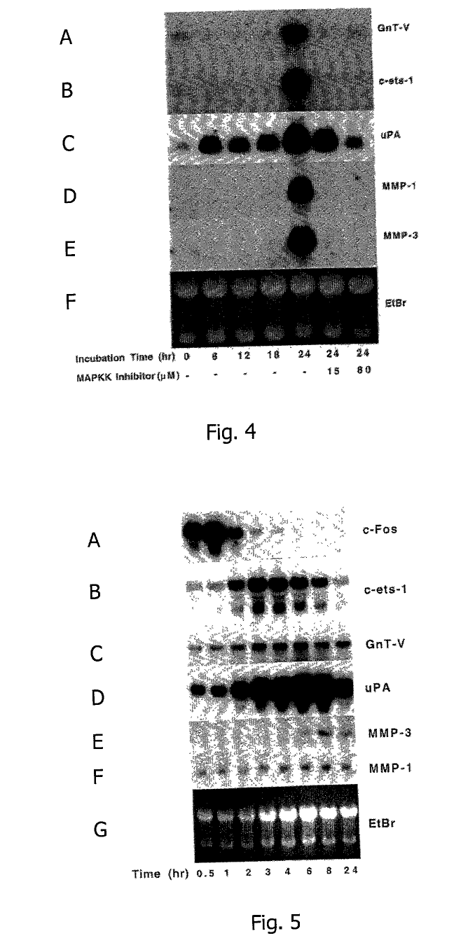

The effects of a test compound, a phorbol ester (a Protein Kinase C (PKC) activator), on the coordinated expression of a panel of sequences was determined. SNB-19 cells were cultured in the presence of 150 nM phorbol 12, 13-dibutyrate (PDBu, Sigma Chemical Co.) for 24 hr. Expression of GnT-V, c-ets-1, uPA, MMP-1, and MMP-3 mRNA was then assayed by northern blot on total RNA from the cells. A panel of nucleic acid probes corresponding to GnT-V, c-ets-1, uPA, MMP-1, and MMP-3 were prepared as described above and utilized to detect expression of the corresponding mRNA in treated or untreated SNB-19 cells. As shown in FIG. 4, mRNA expression for each member of the panel (MMP-1, MMP-3, GnT-V, and c-ets-1) was induced following a 24-hr exposure to PDBu. Thus, the assay identified PDBu as a compound that induces expression of GnT-V, c-ets-1, uPA, MMP-1, and MMP-3 in glioma cells, and therefore a compound that may induce tumor cell activity.

B. Assay for Identification of Compounds that Inhibit Gene Expression in Cancer Cells.

The assay was modified for use in the identification of compounds that can inhibit gene expression. To identify compounds that may affect the growth, migration or invasivity of tumor cells, the assay described below is provided. To activate the quiescent cells, SNB-19 glioma cells are cultured in the presence of 150 nM Phorbol 12, 13-dibutyrate (PDBu, Sigma Chemical Co.) for 24 hr. The cells are then contacted with a test compound that may affect expression of GnT-V, c-ets-1, uPA, MMP-1, or MMP-3. Compounds shown to inhibit expression of any, multiple, or all of the sequences are selected for further study.

An example of such a system is shown below. SNB-19 cells were pre-incubated in the presence of 150 nM Phorbol 12, 13-dibutyrate (PDBu, Sigma Chemical Co.) for 24 hr. The PDBu was then washed out of the culture medium by exchanging the media containing PDBu with fresh media. The pre-incubated cells were then contacted with 15 μM 2′-amino-3′-methoxyflavone, a MAPKK inhibitor (PD 98059, Calbiochem; Alessi, supra). The compound was incubated with the cells for 24 hours and the cells were harvested. Total RNA was then isolated from the cells and a northern blot was performed. A panel of probes corresponding to GnT-V, c-ets-1, uPA, MMP-1 and MMP-3 were prepared and utilized to detect MRNA expression in SB-19 cells. The coordinated induction of GnT-V, c-ets-1, MMP-1, and MMP-3 transcription induced by PDBu was completely abolished by PD 98059 (FIG. 5). uPA expression was decreased in a dose-dependent manner (FIG. 4). Thus, a compound having the ability to affect gene expression in phorbol ester-stimulated cells was identified.

This assay is useful for the identification of other compounds that inhibit expression of GNT-V, c-ets-1, uPA, MMP-1 or MMP-3. A compound that is found to inhibit expression of one or more of these sequences can be selected as a candidate compound for inhibition of tumor activity, such as invasion, in a patient. In such a case, the compound is likely to be a useful candidate for further study as a cancer treatment agent

### Example 3

### Induction of c-Fos by Mitogenic Stimulation

A. Growth-stimulated induction of c-Fos and invasion-related protein mRNAs

Mitogenic stimulation of glioma cells also results in an induction of coordinated gene expression. Quiescent SNB-19 cells were plated in fresh growth media, which resulted in rapid induction of c-Fos mRNA expression, followed by increased expression of c-ets-1 mRNA. This transcription factor expression led to increased expression of uPA, GnT-V, and MMP-3 mRNAs, but not the mRNA of MMP-1 (FIG. 5). MMP-1 transcription therefore requires additional factors. Induction of c-ets-1 resulting from a SNB-19 glioma cell stably transfected with an inducible c-ets-1 gene also resulted in increased GnT-V mRNA expression.

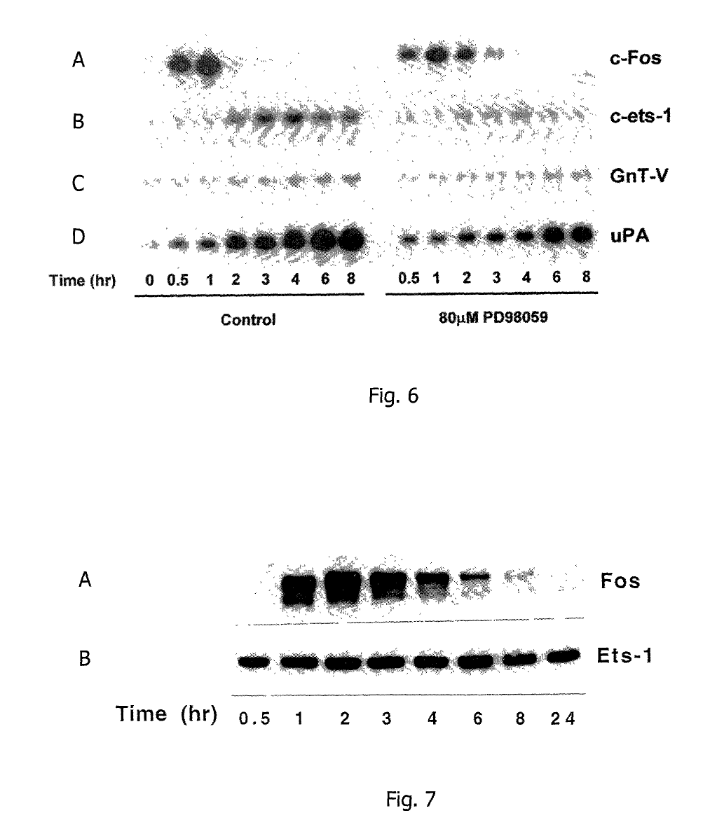

Modulators of the MAPK pathway also modulated the growth-stimulated mRNA expression of c-Fos and invasion-related protein MRNA expression. Quiescent SNB-19 cells were plated in fresh growth media either in the presence of 80 μM PD98059, a MEK/MAPKK inhibitor, or the absence of inhibitor, as a control. Induction was measured by observing MRNA expression over time (FIG. 6). PD98059, an inhibitor of one of the components of the MAPK pathway, also suppressed growth-stimulated induction of c-Fos and invasion-related protein mRNA expression.

B. Growth-stimulated Induction of c-Fos Protein Expression

In SNB-19 glioma cells, c-Fos protein was induced by growth stimulation, whereas levels of c-Ets-1 protein expression remained consistent, despite induction of c-ets-1 mRNA expression (FIG. 7). This result indicates that the c-fos promoter is preferable to the c-ets-1 promoter, for use with a reporter gene to monitor the MAPK activation of SNB-19 glioma cells.

The underlying molecular mechanism of transcriptional control of the invasion-associated genes was also examined using inducible ets-1 transfected SNB-19 glioma cells. Induction of c-Ets-1 protein expression in inducible ets-1 transfected SNB-19 glioma cells resulted in increased expression of uPA MRNA independent of the level of AP-1; whereas expression of GNT-V mRNA was dependent on AP-1 (data not shown). MMP-1 and MMP-3 MRNA expression was not increased by the induction of Ets-1 protein alone. These results indicate that activation of the MAP kinase pathway plays a major role in coordinated expression of invasion-associated genes in gliomas.

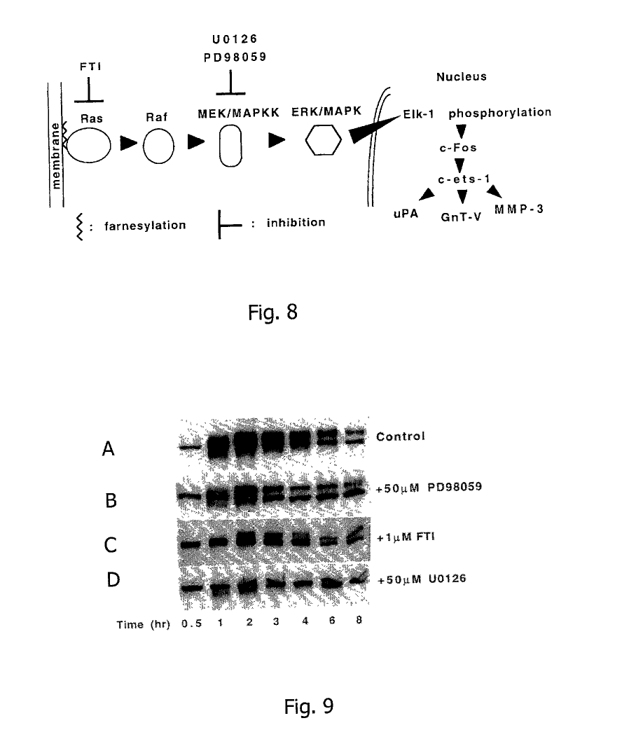

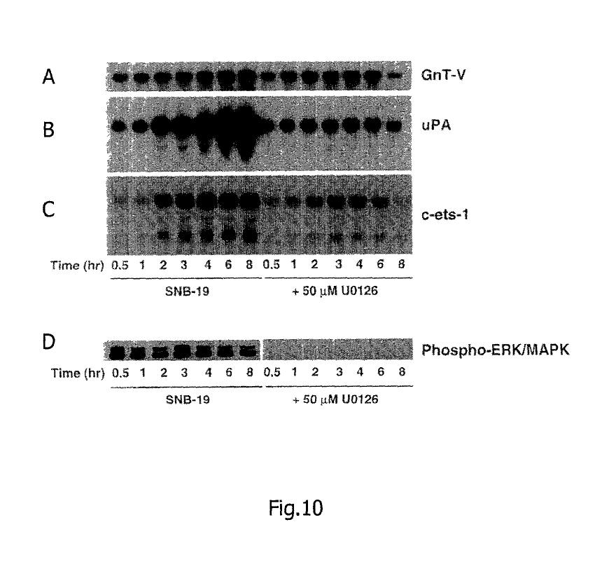

Inhibitors of the MAPK pathway were used to examine the effect of MAPK inhibition on expression of c-Fos protein and the invasion-associated genes in SNB-19 gliomas, in order to determine whether the c-fos promoter would be a good candidate to monitor MAPK activation (FIG. 8). Expression of c-Fos protein was suppressed both by the MEK/MAPKK inhibitors, PD98059 and U0126, and by a farnesyltransferase inhibitor (FTI) (FIG. 9). While U0126 completely inhibited c-Fos protein induction, PD98059 showed weak suppression of c-Fos protein induction. The difference is consistent with the potency of each of the MEK/MAPKK inhibitor. FTI showed moderate inhibition. In addition, U0126 completely inhibited the phosphorylation of ERK/MAPK and suppressed the induction of invasion-associated gene expression (FIG. 10). On the other hand, U0126 had little effect on adhesion-mediated protein tyrosine phosphorylation (data not shown).

These data indicate that modulation of the MAPK pathway can be monitored by c-Fos protein expression in SNB-19 glioma cells. By placing the luciferase gene under control of the c-fos promoter, MAPK pathway activity can be monitored by measuring luciferase enzyme activity as shown in FIG. 1. Likewise, inhibition of the pathway will result in inhibition of luciferase protein expression and, thus, decreased luminescence. Taken together, the luciferase gene placed under control of the c-fos promoter can be used as a reporter to screen inhibitors of the MAPK pathway.

### Example 4

### Induction of c-Fos in SNB-19 Glioma Cells is Specific to the MAPK Pathway

The cross talk between different signal transduction pathways is well known in cancer cells. For example, a signal transduction molecule, Ras, plays a major role in multiple signal transduction pathways, including MAPK and P13K. Amplification and over-expression of EGF receptor also play a role in glioma proliferation and survival in gliomas. Therefore, if the c-fos promoter is to be used to identify modulators of the MAPK pathway, it is necessary to establish that induction of c-fos expression is specific to the MAPK pathway in SNB-19 glioma cells if the c-fos promoter is to be used to identify modulators of the MAPK pathway.

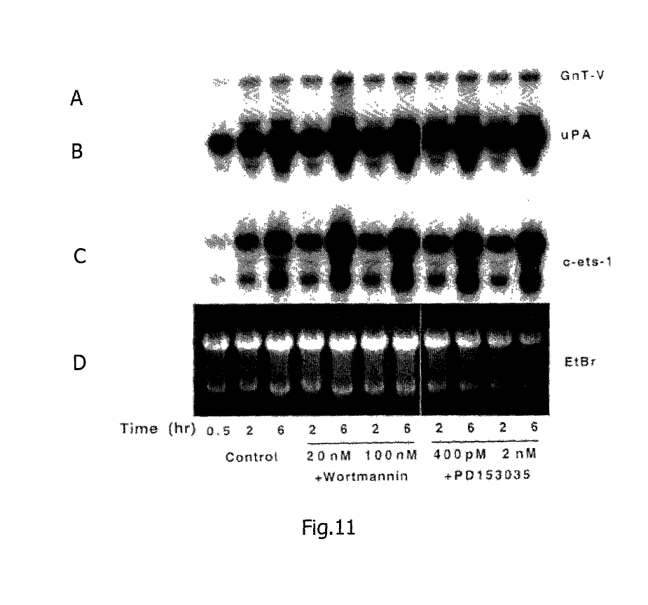

Quiescent SNB-19 cells were growth induced by plating into fresh growth media for 2 and 6 hr. c-Fos expression was monitored by observing the mRNA levels of the downstream genes uPA, GNT-V, and c-ets-1 (see FIG. 8). Unlike MEK/MAPKK inhibitors (Figure), inhibitors of Phoshatidyl inositol 3-kinase (PI 3-kinase) and epidermal growth factor-receptor (EGF-R) kinase, Wortmannin and PD153035 respectively, showed no effect on the expression of uPA, GNT-V, or c-ets-1 mRNA levels in SNB-19 glioma cells under there experimental conditions (FIG. 11).

### Example 5

A. Stable Transfection of the c-Fos Promoter/luciferase Gene with the Bovine Growth Hormone Polyadenylation Tail

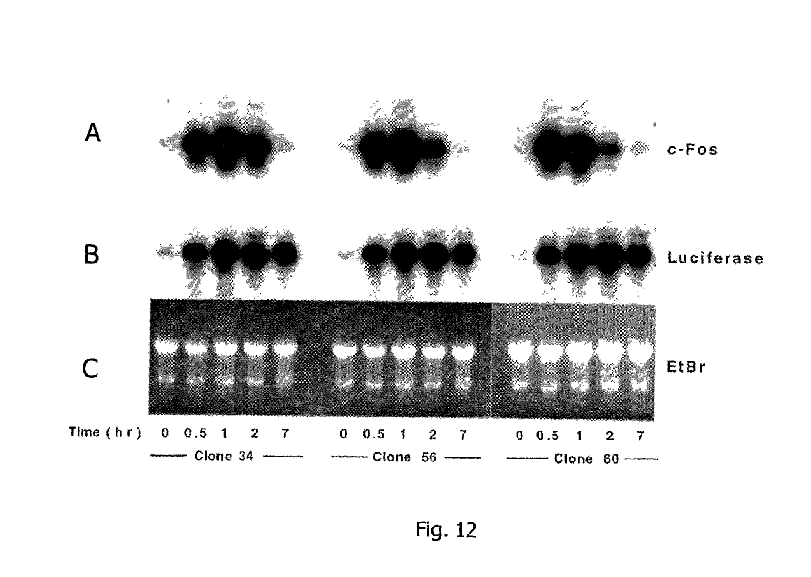

The 498 bp promoter region of the human c-Fos gene was amplified by genomic PCR using sense primer 5′-GTGCGAATGTTCTCTCTCATTCTG-3′ (SEQ ID No. 7) and antisense primer 5′-GCTCAGTCTTGGCTTCTCAGTTG-3′ (SEQ ID No. 8), ligated into the pCR II vector (Invitrogen, San Diego, Calif.). The sequence and orientation for the gene were confirmed by DNA sequencing. Antisense orientation of the c-fos promoter/pCR II plasmid was digested with Hind III and Xho I restriction enzymes and the digested fragment was ligated into the Hind III and Xho I sites of the luciferase vector pGL3-basic (Promega, Madison, Wis.). The c-fos promoter/pGL3 plasmid DNA was digested with Sma I and Xba I and the digested fragment was ligated into the Nru I and Xba I sites of the pcDNA 3 vector (Invitrogen). This final construct contains the luciferase gene under control of the c-fos promoter instead of the control of the CMV promoter. The plasmid DNA was stably transfected into human glioma SNB-19 cells using the cationic liposome system, DOTAP (Boehringer Mannheim, Indianapolis, Ind.). The transfected cells were further cultured for 3 weeks in selection medium containing 800 μg/ml of G418 and then individual clones were isolated with cloning rings. Isolated clones were further cultured for 4 weeks in selection medium and were examined for luciferase gene expression by Northern analysis. Out of 70 clones, 10 clones showed luciferase gene expression. Three representative clones (34, 56, and 60) were further characterized. Growth-stimulated transcription of the luciferase gene correlates to transcription of c-Fos in the clones (FIG. 12). Initiation of luciferase gene transcription starts 30 min post-growth stimulation, similar to that of c-Fos. The expression of luciferase mRNA, however, lasts for a longer period than that for c-Fos mRNA (FIG. 12).

B. Stable Transfection of the c-Fos Promoter/luciferase Gene with the c-Fos 3′untranslated region

Induction of luciferase gene transcription under the c-fos promoter occurred concomitant to that of the c-fos gene upon growth stimulation by serum in the stable transfectant. The expression of luciferase MRNA lasted more than 24 hr, whereas expression of c-fos mRNA diminished within 3 h. Further investigation revealed that this prolonged stability of the luciferase mRNA is apparently due to the bovine growth hormone polyadenylation sequence found in the pcDNA3 vector. In order to allow the time course of the expression of the luciferase gene to correspond more closely to the time course of c-fos gene expression, a construct was created in which the bovine growth hormone polyadenylation sequence is replaced with at least a portion of the c-fos 3 ′-untranslated region (UTR) sequence. Such an insertion of a portion of the 3′-UTR sequence produces constructs in which the time course of the presence of the reporter mRNA under the control of the c-fos promoter more closely corresponds to the time course of the presence of c-fos MRNA under the same conditions.

The 3′ UTR of human c-fos was amplified by RT-PCR using sense primer 5′-TCTAGAGGGGGCAGGGAAGGGGA-3′ (SEQ ID No. 9) (bp 2730 to bp 2746, with the Xba I site at the 51 end) and antisense primer 5′-GCTATTGTCTTCTTTATTGACAATGTCTTGGAACA-3′ (SEQ ID No.10) (bp 3515 to bp 3493, with the Bbs I site at the 5′ end). First, total RNA from SNB-19 cells that were serum stimulated for 1 hr was isolated, and cDNA was synthesized with the antisense primer using Superscript reverse transcriptase (Life Technologies, Rockville, Md.). The 3′-UTR sequences was amplified by PCR using both primers, and the 804 bp product was ligated into the pCR2. 1 vector (Invitrogen, San Diego, Calif.). The sequence and orientation of the DNA was confirmed by DNA sequencing.

The 3-UTR sequence was then cloned into the pcDNA 3 vector. First, the pCR2. 1 plasmid containing the amplified fragment was digested with Xba I and Bbs I. This resulted in two fragments, due to a second Bbs I site present in the amplified fragment. The second Bbs I did not contain the same recogition sequence, and therefore could not be ligiated with the Bbs I site found on the pcDNA 3 vector. The two fragments were ligated into the pcDNA 3 vector that had been digested with Xba I and Bbs I. The sequence of the DNA was confirmed by DNA sequencing. The resulting vector had the c-fos 3′-UTR sequence substituted for the bovine growth hormone polyadenylation sequence.

The newly constructed vector was used in the cloning strategy with the c-fos promoter/pGL3 plasmid as described above. Specifically, the c-fos promoter/pGL3 plasmid was digested with Sma I and Xba I, and the resulting fragment was ligated into the pcDNA 3/c-fos 3′ UTR vector digested with Nru I and Xba I. The resulting vector contains the luciferase gene under the control of the c-fos promoter and the entire c-fos 3′-UTR sequence.

The c-fos promoter/luciferase/c-fos 3′ UTR plasmid was stably transfected into human glioma SNB-19 cells using the cationic liposome system, DOTAP (Boehringer Mannheim, Indianapolis, Ind.). The transfected cells were further cultured for 3 weeks in selection medium containing 800 μg/ml of G418 and then individual clones were isolated with cloning rings. Isolated clones were further cultured for 4 weeks in selection medium and were examined for expression of luciferase mRNA and c-fos 3′-UTR by Northern analysis.

### Example 6

### c-fos Promoter-linked Luciferase Gene Expression

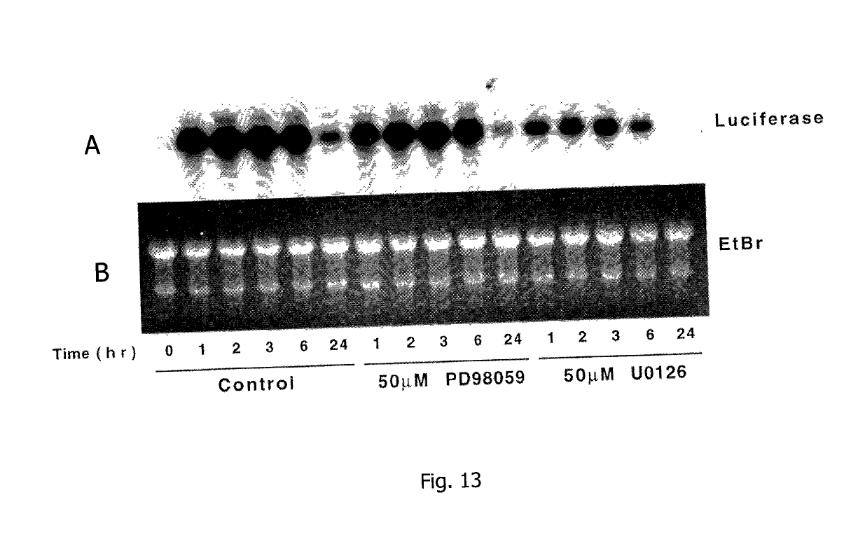

The suppression of luciferase gene expression by inhibitors of MAPK pathway in the stable transfectants was determined. MEK/MAPKK inhibitors (PD98056 and U0126) suppress the expression of the luciferase gene in the stable transfectants (FIG. 13). Levels of inhibition by each inhibitor were consistent with the potency of each inhibitor. For example, incubation with U0126, a potent MEK/MAPKK inhibitor, resulted in greater inhibition of luciferase mRNA expression than did incubation with PD98056, a weak inhibitor. In addition, western-blot analysis with α-luciferase is used to confirm that luciferase protein levels were also inhibited in the presence of MEK/MAPKK inhibitors. Thus, the luciferase gene placed under control of the c-fos promoter can be used as a reporter to screen inhibitors of the MAPK pathway, because luciferase gene expression has been shown to correlate to luciferase protein expression.

### Example 7

### High Throughput Assay to Identify Modulators of MAPK Pathway

A. Procedure for Luciferase Assay

A luminometer, with either a 96-well or 386-well format, is used in a time-course study of luciferase activity to identify compounds that modulate luciferase activity, and thereby modulate the MAPK pathway. Luciferase-transfected cells are cultured to confluency in DMEM containing 10% FBS. The cells are maintained for an additional 4 days post-confluence without culture medium change. The cells are then trypsinized, followed by trypsin neutralization with 2% BSA in PBS. Cells are then harvested according to standard cell culture procedures.

The harvested cells are resupsended in DMEM containing 10% FBS. Approximately 1×10³ to 5×10³ cells are plated in 96-well (or 386-well) plates in the presence of various inhibitors (total volume of 100 pl). After incubation at 37 ° C. for 2-24 hrs in a CO₂ incubator, 100 μl of Bright-Glo reagent (Promega) is added to each well. The plate is then placed into the Reporter Microplate Luminometer (Turner Design). After 5 min and complete cell lysis, luminescence in each well is measured. To measure the effect of inhibitors, cells plated in the absence of inhibitors are used as a negative control (no inhibitors), and cells incubated with 50 μM U0126 are used as a positive control (nearly 100% inhibition). Those skilled in the art will recognize that different compounds can be substituted for both positive and negative controls for both inhibition and activation.

B. High Throughput Assay Utilizing Luciferase Enzyme Activity

Modulators of the MAPK pathway are identified in a high throughput manner by measuring the change in luciferase enzyme activity in c-fos promoter/luciferase gene-transfected SNB-19 glioma cells. As a starting point, a time-course of luciferase enzyme activity is determined. It is known that maximal c-fos mRNA expression is detected at 1 hr post growth-stimulation, and maximal c-Fos protein expression is seen at 2 hr post growth-stimulation (see FIGS. 5, 6, and 9).

In order to establish a high-throughput assay to identify modulators of the MAPK pathway, luciferase activity is determined under different experimental conditions. First, luciferase protein expression and enzyme activity is investigated over a 24 h period to determine the optimal number of cells and incubation time for each assay. Next, complete inhibition of luciferase enzyme activity after incubation with U0126, the potent MEK/MAPKK inhibitor, provides information about the maximal inhibitory effect of MEK/MAPKK phosphorylation, and therefore maximal inhibition of luciferase enzyme activity. Additional experiments include determining dose-response curves for modulators of the MAPK pathway, including, but not limited to, PD98059, FTI, and U0126. The test compounds are assayed using a 1000-fold concentration range to determine the quantitative capacity of the assay system.

Compounds that are known to exhibit different know properties and action mechanisms are also tested. For example, nuclear remodeling is reported to be a downstream event of MAPK activation and plays a major rule in transcription of early response genes such as c-fos. Therefore, the compounds that are tested include modulators of enzymes involving nuclear remodeling, including, but not limited to, histone acetylase, histone deacetylase, histone kinase and phosphorylase. In addition, the activation of Ras is mediated by post-translational modification, including farnesylation and GTP-binding. Therefore, inhibitors of GTP-binding are also tested in this assay.

What is claimed is:

1. A method for identifying a compound affecting the MAPK pathway comprising the steps of:
a) providing a cell stably transfected with a recombinant construct, said recombinant construct comprising a polynucleotide encoding a reporter gene, wherein said reporter gene operatively linked to the c-fos promoter;
b) contacting said cell with a compound; and
c) detecting a change in expression of said reporter gene; whereby a compound affecting the MAPK pathway is identified by detecting the change in expression of said reporter gene under control of the c-fos promoter.

2. A method of claim 1, wherein said reporter gene is the luciferase gene.

3. A method of claim 1 or 2, wherein said compound inhibits the MAPK pathway.

4. A method of claim 1 or 2, wherein said compound activates the MAPK pathway.

5. A method of claim 3, wherein said cell constitutively expresses low levels of invasion-associated genes, whereby stimulation of said invasion-associated genes occurs via activation of the MAPK pathway.

6. A method of claim 4, wherein said cell constitutively expresses low levels of invasion-associated genes, whereby stimulation of said invasion-associated genes occurs via activation of the MAPK pathway.

7. A method of claim 3, wherein said cell is weakly tumorigenic, whereby c-ets-1 mRNA expression is activated exclusively via the MAPK pathway in said cell.

8. A method of claim 4, wherein said cell is weakly tumorigenic, whereby c-ets-1 mRNA expression is activated exclusively via the MAPK pathway in said cell.

9. A method of claim 3, wherein said cell is a SNB-19 glioma cell.

10. A method of claim 4, wherein said cell is a SNB-19 glioma cell.

11. A method of claim 1, wherein the change in expression of said reporter gene is detected as a change in said reporter gene mRNA expression.

12. A method of claim 1, wherein the change in expression of said reporter gene is detected as a change in said reporter gene protein expression.

13. A method of claim 1, wherein the change in expression of said reporter gene is detected as a change in said reporter gene enzyme activity.

14. The method of claim 1, wherein the time course of the change in expression of said reporter gene corresponds to the time course of a change in c-fos gene expression.

15. The method of claim 1, wherein said recombinant construct further comprises at least a portion of the c-fos 3′-untranslated region sequence downstream of said reporter gene sufficient to cause the change in reporter gene expression to correspond to the change in the expression of c-fos.

16. A cell comprising a recombinant construct responsive to modulators of the MAPK pathway, wherein said recombinant construct comprises a polynucleotide encoding a reporter gene operatively linked to the c-fos promoter.

17. A cell of claim 16, wherein said reporter gene is the luciferase gene.

18. A cell of claim 16 or 17, wherein said recombinant construct is responsive to inhibitors of the MAPK pathway.

19. A cell of claim 16 or 17, wherein said recombinant construct is responsive to activators of the MAPK pathway.

20. A cell of claim 16 or 17, wherein said cell constitutively expresses low levels of invasion-associated genes, whereby stimulation of said invasion-associated genes occurs via activation of the MAPK pathway.

21. A cell of claim 16 or 17, wherein said cell is weakly tumorigenic, whereby c-ets-1 mRNA expression is activated exclusively via the MAPK pathway in said cell.

22. A cell of claim 16 or 17, wherein said cell is a SNB-19 glioma cell.

23. A cell of claim 16 or 17, wherein the time course of a change of expression of said reporter gene corresponds to the time course of a change in c-fos expression.

24. A cell of claim 16 or 17, wherein said recombinant construct further comprises at least a portion of the c-fos 3′-untranslated region sequence downstream of said reporter gene sufficient to cause reporter gene expression to correspond to the expression of c-fos.
